(19) United States
(12) Patent Application Publication
(10) Pub. No.: US 2010/0143437 A1
(43) Pub. Date: Jun. 10, 2010

(54) IMPLANTABLE ANALGESIC

(76) Inventors: Edward J. Morris, Bloomington, IN (US); W. Herbert Senft, Martinsville, IN (US)

Correspondence Address: BAKER & DANIELS LLP, 300 NORTH MERIDIAN STREET, SUITE 2700, INDIANAPOLIS, IN, 46204, US

(21) Appl. No.: 12/634,306

(22) Filed: Dec. 9, 2009

Related U.S. Application Data
(60) Provisional application No. 61/121,151, filed on Dec. 9, 2008.

(51) Int. Cl. A61K 9/00; A61P 29/00; A61K 9/48

(52) U.S. Cl. 424/422; 424/400; 424/451

(57) ABSTRACT

A method and device for implantable analgesics having a plurality of analgesia containing portions that are capable of being sequentially released.

### PRIORITY

The present application claims priority to U.S. Provisional Application No. 61/121,151, the disclosure of which is incorporated herein by reference.

### FIELD

The present disclosure relates generally to the administration of analgesic agents to patients. More particularly, the present disclosure relates to a device and method that provides for implantable analgesics for controlled release.

### BACKGROUND AND SUMMARY

Drug delivery devices are used to provide medications or other biologically active substances into human or animal subjects. It is occasionally desired to provide medication to a particular site in the anatomy of the subject. Such delivery can be achieved through a systemic application of the medication that thereby arrives at the desired location. Additionally, fluid based medications can be delivered to the site via injection when access to the site can be achieved.

According to one aspect of the present disclosure, an implantable drug delivery vessel is provided. The vessel including a first portion having a first concentration of a first drug therein, a second portion having a second concentration of a second drug therein, a first biodegradable medium containing the first drug; and a second biodegradable medium containing the second drug; the first and second biodegradable media being placed and composed such that when exposed to an internal portion of a body of the patient, the first and second media successively release the first drug in the first concentration and the second drug in the second concentration.

According to another aspect of the present disclosure, a method for providing sustained local anesthesia to a site in a subject is provided. The method comprising administering to the site a composition comprising a delivery vessel comprising a first anesthetic containing section, and a second anesthetic containing section completely surrounded by the first anesthetic containing section, wherein the first anesthetic containing section, when administered at the site degrades to release anesthetic and to expose the second anesthetic containing section.

According to another aspect of the present disclosure, a biodegradable multi-layered vessel is provided. The vessel comprising anesthetic, and a biocompatible, biodegradable polymer, degradation of a first layer of the biodegradable polymer releasing a first portion of the anesthetic and enables degradation of a second layer of the biodegradable polymer to release a second portion of the anesthetic, the second portion of the anesthetic being distinct from the first portion of the anesthetic and having a defined boundary therebetween.

Additional features of the present disclosure will become apparent to those skilled in the art upon consideration of the following detailed description of the presently perceived best mode of carrying out the disclosure.

### BRIEF DESCRIPTION OF THE DRAWINGS

The detailed description of the drawings particularly refers to the accompanying figures in which:

FIGS. 1 and 1a are side and cross-sectional views of a first embodiment implantable vessel for drug delivery;

FIG. 2 is a cross-sectional view of a second embodiment implantable vessel for drug delivery;

FIG. 3 is a top view of a flat sheet of material of a third embodiment implantable vessel for drug delivery;

FIG. 4 is a perspective view of the sheet of FIG. 3 that has been rolled up.

### DETAILED DESCRIPTION OF THE DRAWINGS

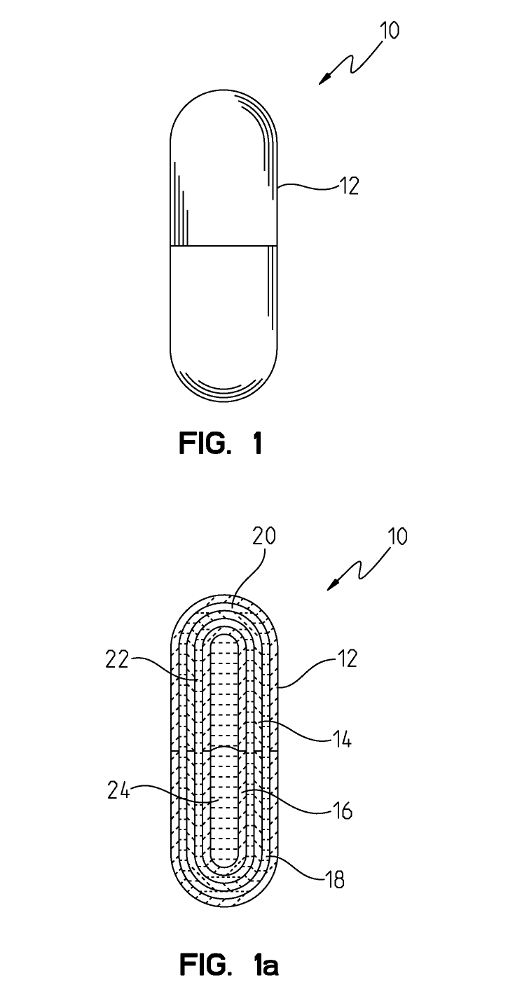

FIG. 1 shows implantable vessel 10. Implantable vessel 10 is formed from multiple dissolvable layers 12, 14, 16, shown in cross section in FIG. 1a, and from liquid analgesic 18 disposed between layers 12, 14, 16.

In the provided example, layers 12, 14, 16 are gelatin capsules that are soluble when placed within tissue of the body but not soluble in liquid analgesic 18. It should be appreciated that alternative embodiments are envisioned where layers 12, 14, 16 are formed from sugar based materials, extracellular matrix based materials, small intestinal submuccosa based materials, urinary bladder matrix based materials, poly-glycolic acid based materials, poly-ethylene based materials, or any other material that provides the desired solubility or analgesic 18 retention and release properties. The material of layers 12, 14, 16 is chosen to provide a desired release schedule. It should also be appreciated that while vessel 10 is shown with three layers 12, 14, 16, more or less layers can be used as desired. For any given composition of layers 12, 14, 16, an increased number of layers will generally be correlated with a longer timeframe necessary to dissolve all layers.

Liquid analgesic 18 is illustratively bupivacaine (1-butyl-N-(2,6-dimethylphenyl) piperidine-2-carboxamide) which has a half life of approximately 210 minutes in adults. It should be appreciated that embodiments are envisioned where other analgesics are used and where the analgesics are in a solid form such as a powder, gel, or otherwise.

In assembly, smaller gelatin capsules are placed within larger gelatin capsules to create layers 12, 14, 16. The capsules thereby define voids 20, 22, 24 between layers 12, 14, 16 and within the innermost layer 16. Bupivacaine 18, or an other analgesic/anesthetic, is placed within each void 20, 22, 24. Embodiments are envisioned where different analgesics or other drugs are placed within different voids 20, 22, 24 of a single vessel 10.

In the present example, a consistent dose of bupivacaine at three regular intervals is desired. Accordingly, bupivacaine of a fist concentration is placed within void 20 that has a first volume. Bupivacaine of a second concentration is placed within void 22. The second concentration is greater than the first concentration in that the volume of void 22 is less than the volume of void 20. The second concentration is set such that even at the lower volume of bupivacaine in void 22, the effective released amount is equal to the effective released amount from void 20. Similarly, because void 24 is larger than both voids 20, 22, the concentration of bupivacaine within void 24 is at a third concentration that is less than the first and second concentrations. Alternatively, bupivacaine of a uniform concentration and is provided to all voids 20, 22, 24 and some voids 20, 24 are not fully filled by the volume of bupivacaine to achieve a uniform effective release amount within each void 20, 22, 24. Furthermore, while the provided example discusses ways to achieve a consistent dose, embodiments are envisioned where a non-consistent dose profile is desired. Accordingly, the concentrations and volumes of bupivacaine within voids 20, 22, 24 can be tailored as desired. Such embodiments where a non-consistent dose profile include, but are not limited to, profiles that are set to release a larger dose during times that the patient is expected to be awake and active relative to the dose to be released during times when the patient is expected to be asleep or otherwise inactive. Each layer 12, 14, 16 and void 20, 22, 24 provides an opportunity to customize the drug and dosing being administered and profiles that desire to taper off the dose being delivered. In a different embodiment, voids 20, 22, 24 contain a gel analgesic. Furthermore, embodiments are envisioned where the gel analgesic is located within a single layer 12. Embodiments are envisioned where the gel contains the analgesic in a homogenous fashion as well as embodiments having a concentration gradient across the capsule to provide areas of differing analgesic concentration.

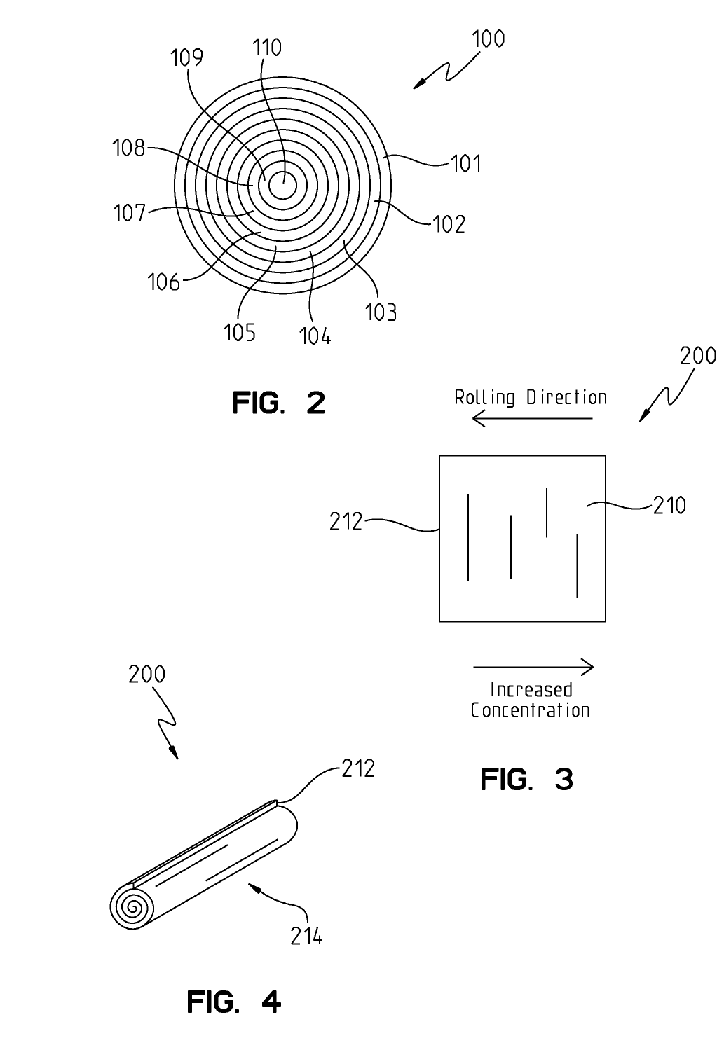

FIG. 2 shows a second embodiment vessel 100 for administering a desired compound, such as bupivacaine. Vessel 100 is substantially spherical and includes multiple solid layers 101-110 as shown in cross section of FIG. 2. As with vessel 10, the number of layers 101-110 can be customized and chosen to produce a desired release profile. Layers 101-110 are formed from a soluble compound that contains bupivacaine. Layers 101-110 are soluble when placed within the body such that the bupivacaine is released to tissue adjacent vessel 100. As with vessel 10, the provided example of vessel 100 includes layers 101-110 having concentrations of bupivacaine that increase from outer layer 101 to layer 110. Therefore, because the volume of layers 101-110 decreases from outer layer 101 to layer 110 the effective delivered dose remains substantially uniform. However, as previously noted, embodiments are envisioned where any desired release profile can be achieved.

In one embodiment, vessel 100 is formed by creating layer 110 and then repeatedly dipping layer 110 in a liquid that later hardens into layer 109. Subsequent layers 101-108 are formed by dipping the existing hardened layers into a liquid that will harden to form the subsequent layers 101-108.

FIG. 3 shows a third embodiment vessel 200 for administering a desired compound, such as bupivacaine. Vessel 200 starts as a single pliable solid sheet 210. Sheet 210 has differing concentration of bupivacaine across its length. FIG. 3 shows sheet 210 having increasing concentration from left to right. Once created, sheet 210 is rolled up, from right to left as shown, and secured in the rolled up state via adhesive or otherwise such that left edge 212 is on an outside of roll 214. This rolling of sheet 210 creates a plurality of layers. As with vessels 10, 100, the number of layers can be customized by customizing the length of sheet 210 to produce a desired release profile. Similarly, sheet 210 can have a continuous concentration gradient across its length or may have discrete areas of differing concentrations that correspond to different layers or otherwise. Sheet 210 is soluble when placed within the body such that the bupivacaine is released to tissue adjacent vessel 200. As with vessels 10, 100, the provided example of vessel 200 includes sheet 210 having concentrations of bupivacaine that increase from left to right in FIG. 3 and from outside to the inside in FIG. 4. Therefore, because the volume of the rolled-up layers decreases from the outer layer to the inner layers the effective delivered dose remains substantially uniform. However, as previously noted, embodiments are envisioned where any desired release profile can be achieved.

In use, vessel 10, 100, 200 is placed within the body and allowed to decompose to elute bipuvacaine or other desired material into the anatomy to provide local analgesia, local anesthesia or nerve blockade. Once vessel 10, 100, 200 is fully decomposed, no part of it remains as a solid within the body. Placement of vessel 10, 100, 200 may accompany a surgery where anatomy suitable for receiving vessel 10, 100, 200 is readily exposed. However, vessel 10, 100, 200 can also be placed independent of the existence of any other contemporary surgical procedure.

Although the disclosure has been described in detail with reference to certain preferred embodiments, variations and modifications exist within the spirit and scope of the disclosure as described and defined in the following claims.

1. An implantable drug delivery vessel including;
a first portion having a first concentration of a first drug therein,
a second portion having a second concentration of a second drug therein,
a first biodegradable medium containing the first drug; and
a second biodegradable medium containing the second drug; the first and second biodegradable media being placed and composed such that when exposed to an internal portion of a body of the patient, the first and second media successively release the first drug in the first concentration and the second drug in the second concentration.

2. The vessel of claim 1, wherein the first drug and the second drug are the same drug.

3. The vessel of claim 1, wherein the first and second biodegradable media are the same drug.

4. The vessel of claim 1, wherein the drug is in a liquid form.

5. The vessel of claim 1, wherein the first and second biodegradable media form first and second capsules to enclose the first and second drugs.

6. The vessel of claim 5, wherein the second capsule is enclosed within the first capsule and the first drug is external to the second capsule.

7. The vessel of claim 1, wherein the first drug is intermixed with the first biodegradable media to form a mixture thereof and the second drug is intermixed with the second biodegradable media to form a mixture thereof.

8. The vessel of claim 1, wherein the first and second concentrations are configured such that a substantially equal amount of anesthetic efficacy is delivered from the first portion and the second portion.

9. A method for providing sustained local anesthesia to a site in a subject, the method comprising:
administering to the site a composition comprising:
a delivery vessel comprising:
a first anesthetic containing section, and
a second anesthetic containing section completely surrounded by the first anesthetic containing section, wherein the first anesthetic containing section, when administered at the site degrades to release anesthetic and to expose the second anesthetic containing section.

10. The method of claim 9, wherein the first anesthetic containing section includes a first capsule and the second anesthetic containing section includes a second capsule and the administering step includes degrading a first biodegradable capsule containing the first anesthetic and the second biodegradable capsule.

11. The method of claim 9, wherein the administering step further includes releasing anesthetic from the second anesthetic containing section subsequent to the release of anesthetic from the first anesthetic containing section.

12. The method of claim 9, wherein the first anesthetic containing section includes a first anesthetic intermixed with a first biodegradable media forming a mixture thereof and the second anesthetic containing section includes a second anesthetic intermixed with a second biodegradable media forming a mixture thereof.

13. The method of claim 9, wherein the administering step includes releasing anesthetic from the first anesthetic containing section and the second anesthetic containing section such that a substantially equal amount of anesthetic efficacy is delivered from each of the anesthetic containing sections.

14. A biodegradable multi-layered vessel comprising:
anesthetic, and
a biocompatible, biodegradable polymer, degradation of a first layer of the biodegradable polymer releasing a first portion of the anesthetic and enables degradation of a second layer of the biodegradable polymer to release a second portion of the anesthetic, the second portion of the anesthetic being distinct from the first portion of the anesthetic and having a defined boundary therebetween.

15. The vessel of claim 14, wherein the boundary is provided by a capsule containing the second portion of the anesthetic.

16. The vessel of claim 14, wherein the boundary is provided by the interface of the first layer having a first composition and the second layer having a second composition that is different from the first composition.

17. The vessel of claim 16, wherein the first composition differs from the second composition by the concentration of the anesthetic therein.

18. The vessel of claim 14, wherein the anesthetic and biocompatible, biodegradable polymer are part of a continuous coiled sheet of material.

19. A method of manufacturing an implantable drug delivery vessel, the method including the steps of;
providing a first biodegradable medium containing a first volume of a first drug of a first concentration,
providing a second biodegradable medium containing a second volume of a second drug of a second concentration, and
surrounding the second biodegradeable medium with the first biodegradable medium such that when exposed to an internal portion of a body of the patient, the first and second media successively release the first drug in the first concentration and the second drug in the second concentration.

20. The method of claim 19, wherein the first volume is greater than the second volume and the first concentration is less than the second concentration.
